(19) United States
(12) Patent Application Publication
(10) Pub. No.: US 2023/0240543 A1
(43) Pub. Date: Aug. 3, 2023

(54) PHYSIOLOGICAL INFORMATION PROCESSING METHOD, PHYSIOLOGICAL INFORMATION PROCESSING APPARATUS, AND STORAGE MEDIUM

(71) Applicant: NIHON KOHDEN CORPORATION, Tokyo (JP)

(72) Inventor: Hironori KUROYONE, Tokorozawa-shi (JP)

(21) Appl. No.: 18/156,695

(22) Filed: Jan. 19, 2023

(30) Foreign Application Priority Data
Jan. 28, 2022 (JP) 2022-011855

(51) Int. Cl. A61B 5/0205; A61B 5/346

(52) U.S. Cl. CPC A61B 5/0205; A61B 5/346; A61B 5/397

(57) ABSTRACT

A physiological information processing method executed by a computer. The physiological information processing method includes acquiring, from a plurality of electrodes attached to a body surface of a subject, a plurality of electrocardiogram signals indicating an electrocardiogram waveform of the subject, separating, through independent component analysis, each of the plurality of electrocardiogram signals into one or more first waveform components indicating the electrocardiogram waveform and one or more second waveform components indicating a waveform other than the electrocardiogram waveform, acquiring an activity evaluation index indicating a respiratory muscle activity amount of the subject based on an electrocardiogram signal that is obtained from a predetermined electrode among the plurality of electrodes attached to the body surface of the subject and from which the one or more first waveform components have been removed, and outputting information on the activity evaluation index.

### CROSS-REFERENCE TO RELATED APPLICATIONS

The present application claims priority from Japanese Patent Application No. 2022-011855, filed on Jan. 28, 2022, the entire content of which is incorporated herein by reference.

### TECHNICAL FIELD

The present disclosure relates to a physiological information processing method and a physiological information processing apparatus configured to evaluate a respiratory muscle activity amount of a subject. Further, the present disclosure relates to a computer-readable storage medium that stores a program for causing a computer to execute the information processing method.

### BACKGROUND

JPH06-30908A discloses a physiological information processing method for acquiring an electromyogram signal indicating a respiratory muscle activity amount based on an electrocardiogram signal using a band-pass filter functioning as a low-cut filter. In general, a signal indicating an electrocardiogram waveform is obtained at a low frequency component of an electrocardiogram signal obtained from an electrocardiogram sensor, and an electromyogram signal is obtained as a noise signal at a high frequency component of the electrocardiogram signal.

Since a frequency band of the electromyogram signal actually extends from a low frequency band to a high frequency band, the frequency band of the electromyogram signal and a frequency band of the electrocardiogram signal partially overlap each other. Therefore, when the electromyogram signal is acquired based on the electrocardiogram signal using the band-pass filter, the low frequency component of the electromyogram signal is removed. As a result, a respiratory muscle activity amount of a subject is evaluated based on only the high frequency component of the electromyogram signal, and thus there is a limit to accuracy of the evaluation of the respiratory muscle activity amount. From the above viewpoint, there is room for examination on a method capable of evaluating the respiratory muscle activity amount of a subject with higher accuracy.

An object of the present disclosure is to provide a physiological information processing method and a physiological information processing apparatus that are capable of evaluating a respiratory muscle activity amount of a subject with higher accuracy. Another object of the present disclosure is to provide a computer-readable storage medium that stores a program for causing a computer to execute the information processing method.

### SUMMARY

According to an aspect of the present disclosure, there is provided a physiological information processing method executed by a computer. The physiological information processing method includes a step of acquiring, from a plurality of electrodes attached to a body surface of a subject, a plurality of electrocardiogram signals indicating an electrocardiogram waveform of the subject, a step of separating, through independent component analysis, each of the plurality of electrocardiogram signals into one or more first waveform components indicating the electrocardiogram waveform and one or more second waveform components indicating a waveform other than the electrocardiogram waveform, a step of acquiring an activity evaluation index indicating a respiratory muscle activity amount of the subject based on an electrocardiogram signal that is obtained from a predetermined electrode among the plurality of electrodes attached to the body surface of the subject and from which the one or more first waveform components have been removed, and a step of outputting information on the activity evaluation index.

According to another aspect of the present disclosure, there is provided a physiological information processing apparatus including one or more processors, and one or more memories configured to store a computer readable command. When the computer readable command is executed by the processor, the physiological information processing apparatus executes a step of acquiring, from a plurality of electrodes attached to a body surface of a subject, a plurality of electrocardiogram signals indicating an electrocardiogram waveform of the subject, a step of separating, through independent component analysis, each of the plurality of electrocardiogram signals into one or more first waveform components indicating the electrocardiogram waveform and one or more second waveform components indicating a waveform other than the electrocardiogram waveform, a step of acquiring an activity evaluation index indicating a respiratory muscle activity amount of the subject based on an electrocardiogram signal that is obtained from a predetermined electrode among the plurality of electrodes attached to the body surface of the subject and from which the one or more first waveform components have been removed, and a step of outputting information on the activity evaluation index.

According to another aspect of the present disclosure, there is provided a non-transitory computer-readable storage medium that stores a program. The program causes a computer to execute a step of acquiring, from a plurality of electrodes attached to a body surface of a subject, a plurality of electrocardiogram signals indicating an electrocardiogram waveform of the subject, a step of separating, through independent component analysis, each of the plurality of electrocardiogram signals into one or more first waveform components indicating the electrocardiogram waveform and one or more second waveform components indicating a waveform other than the electrocardiogram waveform, a step of acquiring an activity evaluation index indicating a respiratory muscle activity amount of the subject based on an electrocardiogram signal that is obtained from a predetermined electrode among the plurality of electrodes attached to the body surface of the subject and from which the one or more first waveform components have been removed, and a step of outputting information on the activity evaluation index.

### BRIEF DESCRIPTION OF DRAWINGS

FIG. 1 is a block diagram illustrating a configuration of a physiological information processing apparatus according to an embodiment (hereinafter, the present embodiment) of the presently disclosed subject matter;

FIG. 2 is a flowchart illustrating a physiological information processing method according to the present embodiment;

FIG. 3 illustrates an example of waveforms of electrocardiogram signals in a 12-lead electrocardiogram examination;

FIG. 4 illustrates electrodes associated with extremity leads;

FIG. 5 illustrates an electrode associated with a chest lead;

FIG. 6 illustrates an example of a waveform component (an electrocardiogram waveform component) indicating an electrocardiogram waveform and a waveform component (a noise waveform component) indicating a waveform other than the electrocardiogram waveform that are separated through independent component analysis;

FIG. 7 illustrates an example of waveforms of 12 types of electrocardiogram signals from which the electrocardiogram waveform component has been removed;

FIG. 8 illustrates a waveform of a difference between an electromyogram signal associated with a V4 lead and an electromyogram signal associated with a V5 lead, on which averaging processing has been executed; and

FIG. 9 illustrates a waveform of the electromyogram signal associated with an aVR lead on which the averaging processing has been executed.

### DESCRIPTION OF EMBODIMENTS

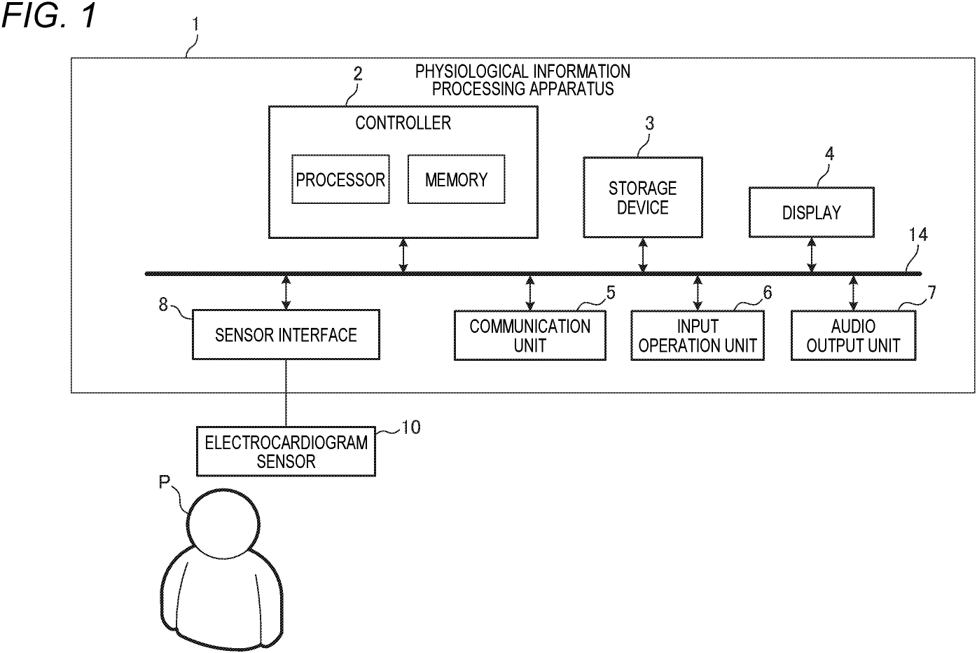

Hereinafter, the present embodiment will be described with reference to the drawings. FIG. 1 is a block diagram illustrating a configuration of a physiological information processing apparatus 1 according to the present embodiment. As illustrated in FIG. 1, the physiological information processing apparatus 1 (hereinafter, simply referred to as the processing apparatus 1) includes a controller 2, a storage device 3, a display 4, a communication unit 5, an input operation unit 6, an audio output unit 7, and a sensor interface 8. These components provided in the processing apparatus 1 are communicably connected to one another via a bus 14.

The processing apparatus 1 may be a medical instrument (for example, a patient monitor) that displays physiological information on a subject P, a personal computer, a workstation, a smartphone, a tablet, or a wearable device (for example, an AR glass) worn on a body (for example, an arm or a head) of a medical worker. The processing apparatus 1 is configured to output information on an activity evaluation index indicating a respiratory muscle activity amount of the subject P.

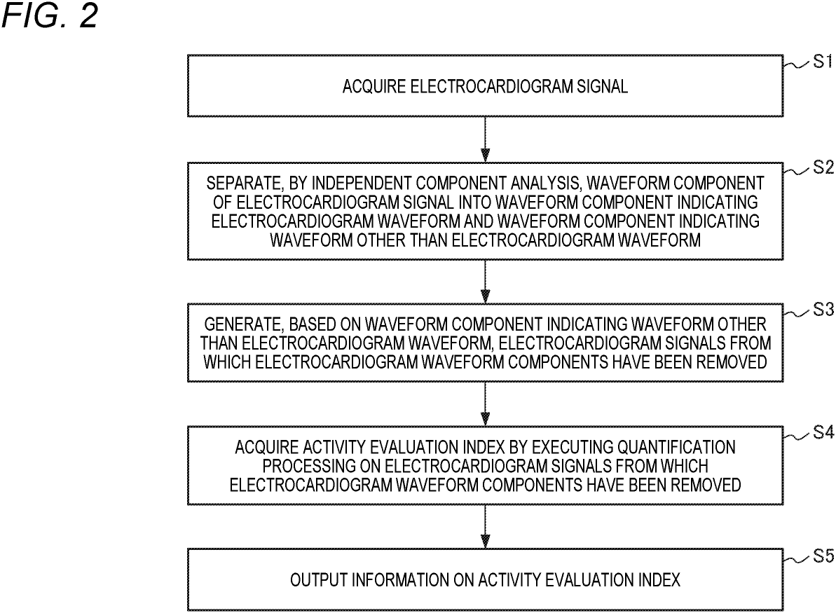

The controller 2 includes one or more processors and one or more memories. The memory is configured to store a computer readable command (a program). The memory can include, for example, a read only memory (ROM) that stores various programs and the like, and a random access memory (RAM) having a plurality of work areas in which various programs and the like to be executed by the processor are stored. The processor includes, for example, at least one of a central processing unit (CPU), a micro processing unit (MPU), and a graphics processing unit (GPU). The CPU may include a plurality of CPU cores. The GPU may include a plurality of GPU cores. The processor may load a designated program from various programs provided in the storage device 3 or the ROM onto the RAM and execute various types of processing in cooperation with the RAM. In particular, the processor loads a physiological information processing program for executing a series of pieces of processing illustrated in FIG. 2 on the RAM, and executes the program in cooperation with the RAM. Details of the physiological information processing program will be described later.

The storage device 3 is, for example, a storage device (a storage) such as a hard disk drive (HDD), a solid state drive (SSD), or a flash memory, and is configured to store a program and various types of data. The physiological information processing program may be provided in the storage device 3. Further, physiological information data (electrocardiogram data or the like) indicating physiological information on the subject P may be stored in the storage device 3. For example, the electrocardiogram data acquired from an electrocardiogram sensor 10 may be stored in the storage device 3 via the sensor interface 8.

The communication unit 5 is configured to connect the processing apparatus 1 to an in-hospital network. Specifically, the communication unit 5 may include various wired connection terminals that communicate with a central monitor or a server provided in the in-hospital network. The communication unit 5 may further include a wireless communication module that performs wireless communication with the central monitor or the server. The communication unit 5 may include, for example, a wireless communication module corresponding to a medical telemeter system. The communication unit 5 may include a wireless communication module corresponding to a wireless communication standard such as Wi-Fi (registered trademark) or Bluetooth (registered trademark) and/or a wireless communication module corresponding to a mobile communication system using a SIM. The in-hospital network may be, for example, a local area network (LAN) or a wide area network (WAN). The processing apparatus 1 may be connected to the Internet via the in-hospital network.

The display 4 is configured to display physiological information (for example, information related to an activity evaluation index) of the subject P, and is, for example, a liquid crystal panel or an organic EL panel. The input operation unit 6 is, for example, a touch panel overlapping the display 4, a mouse, and/or a keyboard. The input operation unit 6 is configured to receive an input operation performed by the medical worker and to generate an operation signal corresponding to the input operation performed by the medical worker. After the operation signal generated by the input operation unit 6 has been transmitted to the controller 2 via the bus 14, the controller 2 executes a predetermined operation according to the operation signal. The audio output unit 7 includes one or more speakers.

The sensor interface 8 is an interface configured to connect the electrocardiogram sensor 10 to the processing apparatus 1. The sensor interface 8 may include an input terminal to which an electrocardiogram signal output from the electrocardiogram sensor 10 is input. The electrocardiogram sensor 10 is configured to acquire an electrocardiogram signal indicating an electrical activity (an electrocardiogram waveform) of a heart of the subject P, and can include a plurality of electrodes attached to a body surface of the subject P. In this example, the electrocardiogram sensor 10 can be applied to a 12-lead electrocardiogram examination. Therefore, the electrocardiogram sensor 10 can include four electrodes (an example of a second electrode) associated with extremity leads and six electrodes (an example of a first electrode) associated with a chest lead.

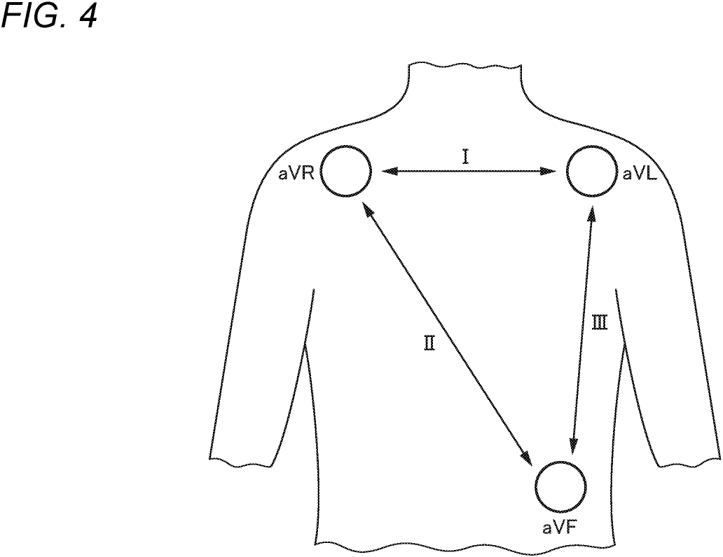

As illustrated in FIG. 4, the electrocardiogram sensor 10 can include, as four electrodes associated with extremity leads, a lead electrode aVR associated with an aVR lead, a lead electrode aVL associated with an aVL lead, a lead electrode aVF associated with an aVF lead, and a ground electrode (not illustrated). The lead electrode aVR is attached to a right hand of the subject P. The lead electrode aVL is attached to a left hand of the subject P. The lead electrode aVF is attached to a left foot of the subject P. The ground electrode is attached to a right foot of the subject P. An electrocardiogram signal associated with a I lead is acquired based on the lead electrodes aVL, aVR. An electrocardiogram signal associated with a III lead is acquired based on the lead electrodes aVL, aVF. An electrocardiogram signal associated with a II lead is acquired based on the lead electrodes aVR, aVF.

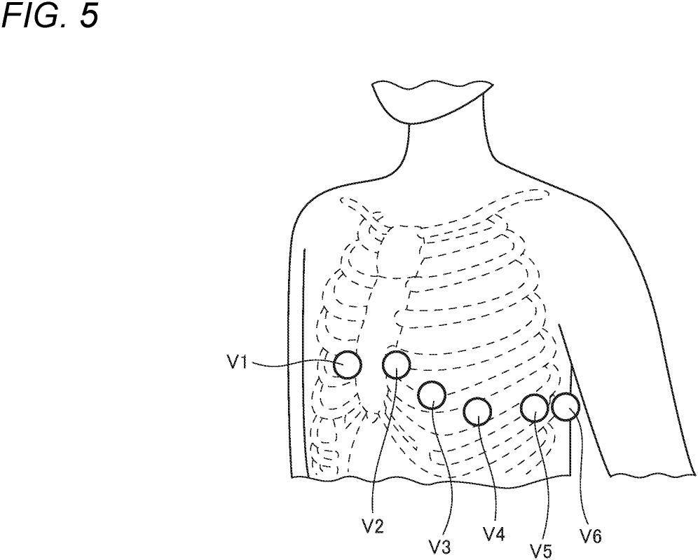

As illustrated in FIG. 5, the electrocardiogram sensor 10 can include lead electrodes V1 to V6 mounted in an intercostal space of the subject P as six electrodes related to chest leads. In the lead electrode V1, an electrocardiogram signal related to a V1 lead is acquired. In the lead electrode V2, an electrocardiogram signal related to a V2 lead is acquired. In the lead electrode V3, an electrocardiogram signal related to a V3 lead is acquired. In the lead electrode V4, an electrocardiogram signal related to a V4 lead is acquired. In the lead electrode V5, an electrocardiogram signal related to a V5 lead is acquired. In the lead electrode V6, an electrocardiogram signal related to a V6 lead is acquired. The lead electrode V1 is attached to a right edge of a fourth intercostal sternum of the subject P. The lead electrode V2 is attached to a left edge of the fourth intercostal sternum of the subject P. The lead electrode V3 is attached to a position of a midpoint on a line connecting the lead electrode V2 and the lead electrode V4. The lead electrode V4 is attached to a position of an intersection of a fifth intercostal space and a left midclavicular line of the subject P. The lead electrode V5 is attached to a position having the same height as the lead electrode V4 on a left anterior axillary line. The lead electrode V6 is attached to a position having the same height as the lead electrode V4 on a left middle axillary line.

The sensor interface 8 can include at least a plurality of differential amplifier circuits and an AD converter. Each of the plurality of differential amplifier circuits is configured to amplify an electrocardiogram signal output from a corresponding lead electrode. The AD converter is configured to convert an electrocardiogram signal from an analog signal to a digital signal. The electrocardiogram signal converted into the digital signal is transmitted from the sensor interface 8 to the controller 2.

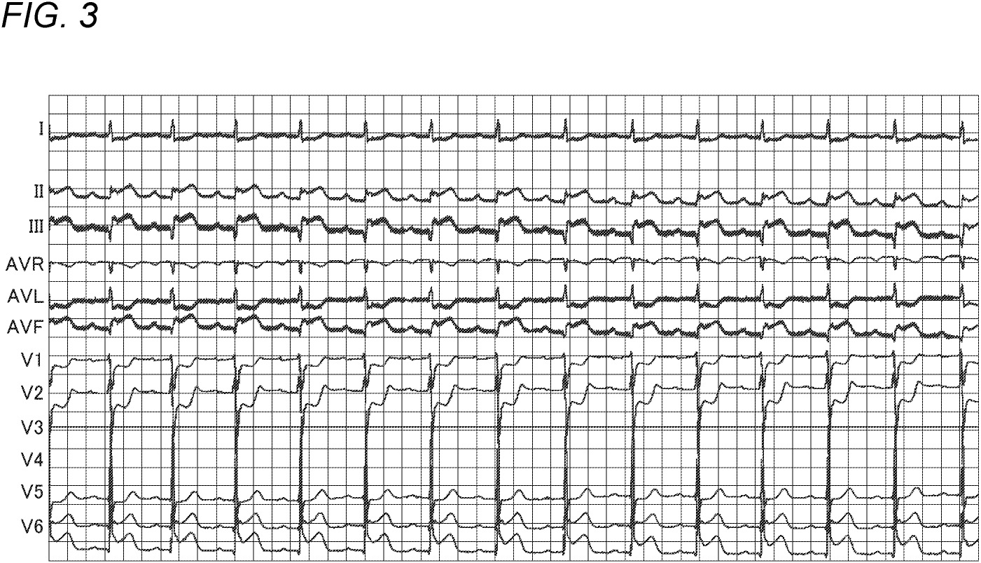

In the present embodiment, the processing apparatus 1 can acquire 12 types of electrocardiogram signals through the 12-lead electrocardiogram examination. In particular, the processing apparatus 1 can acquire three electrocardiogram signals associated with standard limb leads, three electrocardiogram signals associated with unipolar limb leads, and six electrocardiogram signals associated with chest leads. As illustrated in FIG. 3, the three electrocardiogram signals associated with the standard limb leads include electrocardiogram signals associated with a I lead, a II lead, and a III lead. The three electrocardiogram signals associated with the unipolar limb leads are electrocardiogram signals associated with an aVR lead, an aVL lead, and an aVF lead. The six electrocardiogram signals associated with the chest leads include electrocardiogram signals associated with the V1 lead, the V2 lead, the V3 lead, the V4 lead, the V5 lead, and the V6 lead.

Next, a physiological information processing method according to the present embodiment will be described below with reference to FIG. 2. FIG. 2 is a flowchart illustrating the physiological information processing method according to the present embodiment.

As illustrated in FIG. 2, in step S1, the controller 2 acquires a plurality of electrocardiogram signals indicating electrocardiographic waveforms of the subject P from the electrocardiogram sensor 10 via the sensor interface 8. In particular, as illustrated in FIG. 3, the controller 2 acquires the 12 types of electrocardiogram signals acquired in the 12-lead electrocardiogram examination as digital signals. As described above, the 12 types of electrocardiogram signals include the electrocardiogram signals associated with the standard limb leads, the electrocardiogram signals associated with the unipolar limb leads, and the electrocardiogram signals associated with the chest leads.

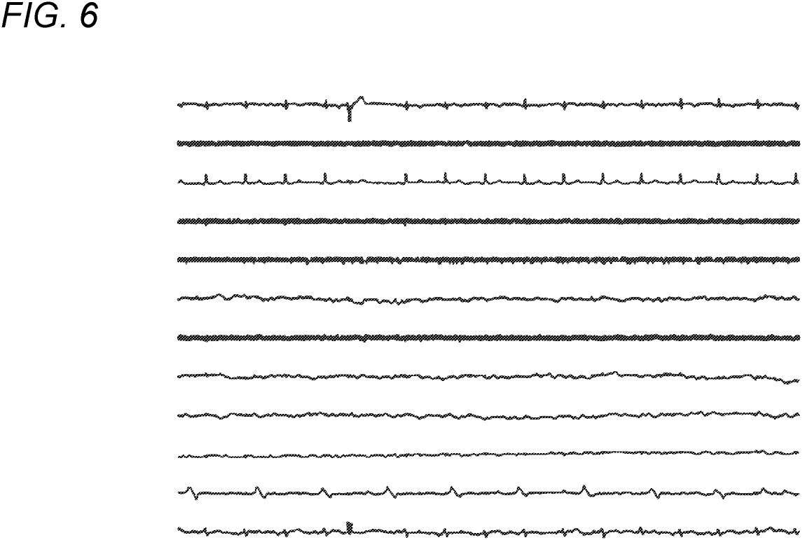

In step S2, the controller 2 separates, by independent component analysis (ICA), a waveform component of each electrocardiogram signal into a waveform component indicating an electrocardiogram waveform and a waveform component indicating a waveform other than the electrocardiogram waveform. The independent component analysis is a calculation method for separating multivariate signals into a plurality of additive components. In the present embodiment, since the 12 types of electrocardiogram signals are simultaneously acquired by the 12-lead electrocardiogram examination, each of the 12 types of electrocardiogram signals can be separated into 12 types of waveform components by the independent component analysis. FIG. 6 shows a waveform component (hereinafter referred to as an electrocardiogram waveform component) indicating an electrocardiogram waveform and a waveform component (hereinafter referred to as a noise waveform component) indicating a waveform other than the electrocardiogram waveform that are separated through the independent component analysis in a predetermined electrocardiogram signal. As illustrated in FIG. 6, the electrocardiogram signal includes a plurality of electrocardiogram waveform components (an example of a first waveform component) and a plurality of noise waveform components (an example of a second waveform component) indicating a respiratory muscle activity.

When an electrocardiogram signal vector x(t) including the 12 types of electrocardiogram signals is x(t) = (x₁(t), ... s₁₂(t))^{T}, and a waveform component vector s(t) including the 12 types of waveform components is (s₁(t), ... s₁₂(t))^{T}, relation between x(t) and s(t) in the independent component analysis is expressed as x(t) = As(t). Here, A is a coefficient matrix formed by 12 rows x 12 columns.

Next, the controller 2 classifies the 12 types of waveform components into an electrocardiogram waveform component and a noise waveform component in each electrocardiogram signal. In this regard, the controller 2 can classify the 12 types of waveform components into the electrocardiogram waveform component and the noise waveform component by comparing an electrocardiogram waveform (hereinafter referred to as a reference electrocardiogram waveform) serving as a reference with each waveform component. For example, when a correlation coefficient between a predetermined waveform component and the reference electrocardiogram waveform is large, the predetermined waveform component may be classified as the electrocardiogram waveform component. On the other hand, when the correlation coefficient between the predetermined waveform component and the reference electrocardiogram waveform is small, the predetermined waveform component may be classified as the noise waveform component.

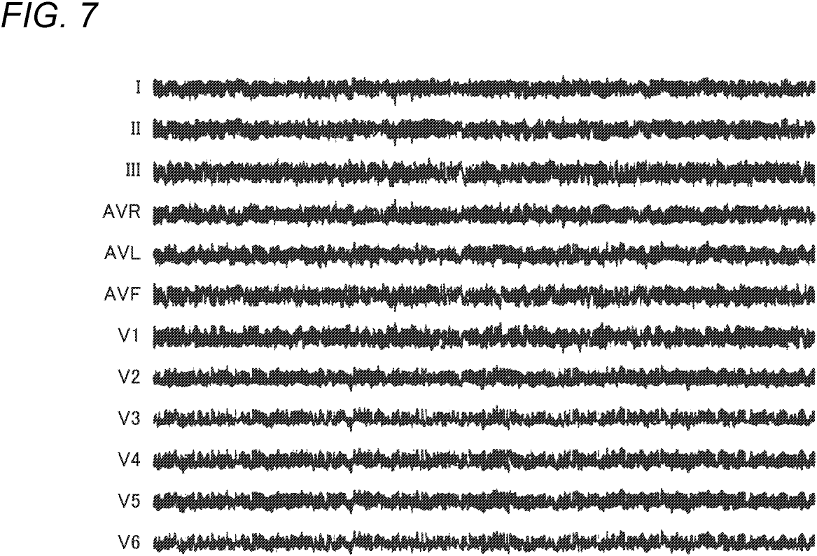

Next, in step S3, the controller 2 generates, based on only the noise waveform component indicating waveforms other than the electrocardiogram waveform, 12 types of electrocardiogram signals from which electrocardiogram waveform components have been removed. As described above, each of the 12 types of electrocardiogram signals includes a plurality of electrocardiogram waveform components and a plurality of noise waveform components. Therefore, the controller 2 generates 12 types of electrocardiogram signals each including only a plurality of noise waveform components such that the electrocardiogram waveform components are removed in each electrocardiogram signal. FIG. 7 illustrates an example of waveforms of the 12 types of electrocardiogram signals from which the electrocardiogram waveform components have been removed.

For example, when the electrocardiogram signal x₁ associated with the V1 lead is expressed by x₁ = a₁₁s₁ + a₁₂s₂ + ... a₁₁₂s₁₂, and s₁, S₂, and S₃ are electrocardiogram waveform components, the electrocardiogram signal x₁′ from which the electrocardiogram waveform components have been removed is expressed as x₁′ = a₁₄S₄ + a₁₅S₅ + ... a₁₁₂S₁₂. In this way, the electrocardiogram waveform component can be removed from each electrocardiogram signal through the independent component analysis. The electrocardiogram signal from which the electrocardiogram waveform components have been removed is treated as an electromyogram signal indicating the respiratory muscle activity amount of the subject P. In this regard, in the related-art electromyogram signal, the low frequency component has been removed, whereas the electromyogram signal acquired in the present embodiment includes both the low frequency component and the high frequency component.

Next, in step S4, the controller 2 executes quantification processing on the electrocardiogram signal from which the electrocardiogram waveform components have been removed to acquire the activity evaluation index indicating the respiratory muscle activity amount of the subject P. Hereinafter, for convenience of description, the electrocardiogram signal from which the electrocardiogram waveform components have been removed is referred to as an electromyogram signal. In this regard, the controller 2 may execute the quantification processing on all of the 12 types of electromyogram signals, or may execute the quantification processing on some of the 12 types of electromyogram signals. In the quantification processing, the controller 2 executes averaging processing on the electromyogram signal, and then determines a maximum amplitude, an average amplitude, or an integrated value of the electromyogram signal subjected to the averaging processing as the activity evaluation index.

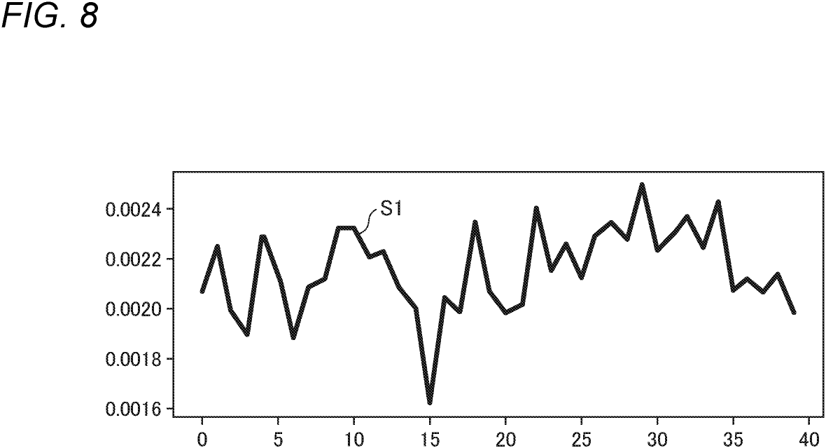

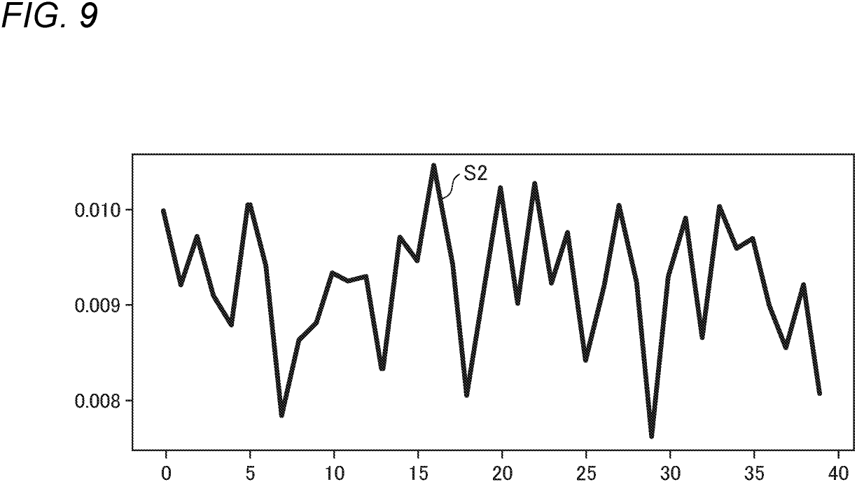

As the averaging processing, root mean square (RMS) processing, interval averaging processing in which absolute value processing is set as preprocessing, integration processing, and the like may be used. A section set in the RMS processing may be, for example, within a range of 100 ms to 300 ms. FIG. 7 illustrates waveforms of 12 types of electromyogram signals before the averaging processing is executed, whereas FIGS. 8 and 9 illustrate examples of the waveforms of the electromyogram signals on which the averaging processing has been executed. The controller 2 can determine, as the activity evaluation index, the maximum amplitude, the average amplitude, or the integrated value of the waveform of the electromyogram signal on which the averaging processing has been executed.

Further, the controller 2 may acquire, as the activity evaluation index indicating the respiratory muscle activity amount, a first activity evaluation index indicating an activity amount of a diaphragm (an example of a first respiratory muscle) of the subject P and a second activity evaluation index indicating an activity amount of an intercostal muscle (an example of a second respiratory muscle) of the subject P.

### First Activity Evaluation Index

The controller 2 may acquire the first activity evaluation index indicating the activity amount of the diaphragm by executing the quantification processing on at least one of the six types of electromyogram signals associated with the chest leads. For example, the controller 2 calculates a difference between the electromyogram signal associated with the V4 lead and the electromyogram signal associated with the V5 lead, and then executes the averaging processing on a waveform of the calculated difference. FIG. 8 illustrates a waveform S1 of the difference between the electromyogram signal associated with the V4 lead and the electromyogram signal associated with the V5 lead, on which the averaging processing has been executed. Thereafter, the controller 2 may determine, as the first activity evaluation index, a maximum amplitude, an average amplitude, or an integrated value of the waveform S1 of the difference on which the averaging processing has been executed. In this example, when the first activity evaluation index is determined, a waveform of a difference between the electromyogram signal associated with the V4 lead and the electromyogram signal associated with the V5 lead is used. However, the present embodiment is not limited thereto. For example, when the first activity evaluation index is determined, a waveform of any one of the electromyogram signals related to the V3 to V6 leads, a waveform of a difference between the electromyogram signal associated with the V5 lead and the electromyogram signal associated with the V6 lead, or a waveform of a difference between the electromyogram signal associated with the V3 lead and the electromyogram signal associated with the V4 lead may be used.

### Second Activity Evaluation Index

The controller 2 may acquire the second activity evaluation index indicating the activity amount of the intercostal muscle by executing the quantification processing on the electromyogram signal associated with the aVR lead. For example, the controller 2 executes the averaging processing on the electromyogram signal associated with the aVR lead. FIG. 9 illustrates a waveform S2 of the electromyogram signal associated with the aVR lead on which the averaging processing has been executed. Thereafter, the controller 2 may determine, as the second activity evaluation index, a maximum amplitude, an average amplitude, or an integrated value of the waveform S2 of the electromyogram signal on which the averaging processing has been executed. In this example, when the second activity evaluation index is determined, the waveform S2 of the electromyogram signal associated with the aVR lead is used. However, the present embodiment is not limited thereto. For example, when the second activity evaluation index is determined, the waveform of the electromyogram signal associated with the aVL lead may be used. A waveform of a difference between the electromyogram signal associated with the aVR lead and the electromyogram signal associated with the V1 lead may be used, or a waveform of a difference between the electromyogram signal associated with the aVL lead and the electromyogram signal associated with the V2 lead may be used.

The inventor of the present disclosure classifies the 12 types of electromyogram signals into four factors through factor analysis, which is a type of the multivariate analysis. As a result of the factor analysis, it has been found that the electromyogram signals associated with the V4 lead, the V5 lead, and V6 lead particularly strongly indicate the activity amount of the diaphragm. It has been found that the electromyogram signals associated with the aVR lead and the aVL lead particularly strongly indicate the activity amount of the intercostal muscle. In this way, in the first activity evaluation index indicating the activity amount of the diaphragm, the electromyogram signals associated with the V4 lead, the V5 lead, and V6 lead are used, whereas in the second activity evaluation index indicating the activity amount of the intercostal muscle, the electromyogram signals associated with the aVR lead and the aVL lead are used.

Next, in step S5, the controller 2 outputs information on the activity evaluation index. In this regard, the controller 2 may display information on the activity evaluation index on a display screen of the display 4. Examples of the information on the activity evaluation index include a current numerical value of the activity evaluation index, a trend graph indicating a temporal change in the activity evaluation index, and an increase or decrease amount or an increase or decrease rate of the activity evaluation index with respect to a predetermined reference value. Here, the predetermined reference value may be determined based on a plurality of acquired activity evaluation indices. The current numerical value of the activity evaluation index, the trend graph of the index, and the increase or decrease amount of the index may be simultaneously displayed on the display screen of the display 4. In particular, the controller 2 may simultaneously display, on the display screen of the display 4, the information on the first activity evaluation index indicating the activity amount of the diaphragm and the information on the second activity evaluation index indicating the activity amount of the intercostal muscle. More specifically, the trend graph of the first activity evaluation index and the trend graph of the second activity evaluation index may be simultaneously displayed on the display screen. In this way, a medical worker can grasp an activity state of the diaphragm of the subject P and an activity state of the intercostal muscle of the subject P by viewing the two trend graphs displayed on the display screen.

The controller 2 may store information on the activity evaluation index in the storage device 3, or may transmit the information to a central monitor or a server provided in the in-hospital network.

According to the present embodiment, the electrocardiogram signal is separated into the electrocardiogram waveform component and the noise waveform component other than the electrocardiogram waveform component through the independent component analysis. Thereafter, the first activity evaluation index indicating the activity amount of the diaphragm and the second activity evaluation index indicating the activity amount of the intercostal muscle are acquired based on the electrocardiogram signal (the electromyogram signal) from which the electrocardiogram waveform component has been removed. Thereafter, the information on the first activity evaluation index and the second activity evaluation index is output. In this way, in the present embodiment, the respiratory muscle activity amount of the subject P is evaluated based on both the low frequency component and the high frequency component of the electromyogram signal, unlike the related-art method for evaluating the respiratory muscle activity amount using a band-pass filter. That is, in the present embodiment, it is possible to acquire the electromyogram signal without removing a specific frequency component. Therefore, it is possible to evaluate the respiratory muscle activity amount of the subject with high accuracy as compared with the related-art method in which the respiratory muscle activity amount of the subject is evaluated based on the electromyogram signal from which the low frequency component has been removed.

The medical worker can grasp the temporal change in the respiratory muscle activity amount of the subject P by viewing the trend graph or the like of the activity evaluation index of the subject P output from the processing apparatus 1. The medical worker can grasp a state (for example, exhaustion or weakening of the respiratory muscle of the diaphragm or the like associated with resting, weighted side lung injury due to a decrease in the activity amount of the diaphragm, an effect of respiratory muscle training, and an activity of a sternocleidomastoid muscle and an accessory respiratory muscle) of the respiratory muscle of the subject P based on the temporal change in the respiratory muscle activity amount of the subject P.

In the present embodiment, the activity evaluation index indicating the respiratory muscle activity amount of the subject P can be acquired using the electrodes used in the 12-lead electrocardiogram examination. In this way, it is possible to evaluate the respiratory muscle activity amount of the subject P with high accuracy while measuring the electrocardiogram of the subject P.

In the present embodiment, the activity amount of the two respiratory muscles which are the diaphragm and the intercostal muscle is evaluated based on the quantified electromyogram signal. However, the present embodiment is not limited thereto. In this regard, the controller 2 may evaluate the activity amount of the accessory respiratory muscle by adding the activity amount of the accessory respiratory muscle to the two activity amounts of the diaphragm and the intercostal muscle. That is, the controller 2 may acquire a third activity evaluation index indicating the activity amount of the accessory respiratory muscle based on the quantified electromyogram signal, and then add the third activity evaluation index to the information on the first activity evaluation index and the second activity evaluation index and output information on the third activity evaluation index.

In order to implement the processing apparatus 1 according to the present embodiment by software, a physiological information processing program may be assembled into the storage device 3 or the ROM in advance. Alternatively, the physiological information processing program may be stored in a computer-readable storage medium such as a magnetic disk (for example, a HDD or a floppy disk), an optical disk (for example, a CD-ROM, a DVD-ROM, or a Blu-ray (registered trademark) disk), a magneto-optical disk (for example, a MO), or a flash memory (for example, a SD card, a USB memory, or a SSD). In this case, the physiological information processing program stored in the storage medium may be assembled into the storage device 3. Further, the program assembled in the storage device 3 may be loaded onto the RAM, and then the processor may execute the program loaded onto the RAM. In this way, the physiological information processing method according to the present embodiment is executed by the processing apparatus 1.

The physiological information processing program may be downloaded from a computer on a communication network via the communication unit 5. In this case, same or similarly, the downloaded program may be assembled into the storage device 3.

According to the present disclosure, it is possible to provide a physiological information processing method and a physiological information processing apparatus that are capable of evaluating a respiratory muscle activity amount of a subject with higher accuracy. It is also possible to provide a program for causing a computer to execute the information processing method and a computer-readable storage medium that stores the program.

According to the physiological information processing method in the present disclosure, through the independent component analysis, the electrocardiogram signal is separated into one or more first waveform components indicating an electrocardiogram waveform and one or more second waveform components indicating a waveform other than the electrocardiogram waveform, and then the respiratory muscle activity evaluation index indicating the activity amount of the respiratory muscle is acquired based on the electrocardiogram signal from which the first waveform component has been removed. In this way, in the above-described method, the respiratory muscle activity amount of the subject is evaluated based on both the low frequency component and the high frequency component of the electromyogram signal (that is, the electrocardiogram signal from which the first waveform component has been removed), unlike the related-art method for evaluating the respiratory muscle activity amount using the band-pass filter. That is, it is possible to evaluate the respiratory muscle activity amount of the subject with high accuracy as compared with the related-art method in which the respiratory muscle activity amount is evaluated based on the electromyogram signal from which the low frequency component has been removed.

According to the physiological information processing apparatus in the present disclosure, it is possible to provide the physiological information processing apparatus capable of evaluating the respiratory muscle activity amount of the subject with high accuracy as compared with the related-art method in which the respiratory muscle activity amount is evaluated based on the electromyogram signal from which the low frequency component has been removed.

The embodiment of the presently disclosed subject matter is described above. However, the technical scope of the presently disclosed subject matter should not be construed as being limited to the description of the embodiment. It is understood by those skilled in the art that the present embodiment is an example and various modifications can be made within the scope of the inventions described in the claims. The technical scope of the presently disclosed subject matter should be determined based on the scope of the invention described in the claims and the scope of equivalents thereof.

What is claimed is:

1. A physiological information processing method executed by a computer, the physiological information processing method comprising:
a step of acquiring, from a plurality of electrodes attached to a body surface of a subject, a plurality of electrocardiogram signals indicating an electrocardiogram waveform of the subject;
a step of separating, through independent component analysis, each of the plurality of electrocardiogram signals into one or more first waveform components indicating the electrocardiogram waveform and one or more second waveform components indicating a waveform other than the electrocardiogram waveform;
a step of acquiring an activity evaluation index indicating a respiratory muscle activity amount of the subject based on an electrocardiogram signal that is obtained from a predetermined electrode among the plurality of electrodes attached to the body surface of the subject and from which the one or more first waveform components have been removed; and
a step of outputting information on the activity evaluation index.

2. The physiological information processing method according to claim 1,
wherein, in the step of acquiring the activity evaluation index, the activity evaluation index is acquired by executing quantification processing on the electrocardiogram signal from which the one or more first waveform components have been removed.

3. The physiological information processing method according to claim 2,
wherein the step of acquiring the activity evaluation index includes
a step of executing, at a predetermined time interval, averaging processing on the electrocardiogram signal from which the one or more first waveform components have been removed, and
a step of determining, as the activity evaluation index, a maximum amplitude, an average amplitude, or an integrated value of the electrocardiogram signal subjected to the averaging processing.

4. The physiological information processing method according to claim 1,
wherein the information on the activity evaluation index includes at least one of a trend graph illustrating a temporal change in the activity evaluation index, and an increase or decrease amount or an increase or decrease rate of the activity evaluation index with respect to a predetermined reference value.

5. The physiological information processing method according to claim 1,
wherein the step of acquiring the activity evaluation index further includes
a step of acquiring a first activity evaluation index indicating an activity amount of a first respiratory muscle based on a first electrocardiogram signal that is obtained from a first electrode among the plurality of electrodes and from which the one or more first waveform components have been removed, and
a step of acquiring a second activity evaluation index indicating an activity amount of a second respiratory muscle different from the first respiratory muscle based on a second electrocardiogram signal that is obtained from a second electrode among the plurality of electrodes and from which the one or more first waveform components have been removed, and
wherein the step of outputting the information on the activity evaluation index includes
a step of outputting information on the first activity evaluation index and information on the second activity evaluation index.

6. The physiological information processing method according to claim 5,
wherein, in the step of acquiring the first activity evaluation index, the first activity evaluation index is acquired by executing quantification processing on the first electrocardiogram signal from which the one or more first waveform components have been removed, and
wherein, in the step of acquiring the second activity evaluation index, the second activity evaluation index is acquired by executing quantification processing on the second electrocardiogram signal from which the one or more first waveform components have been removed.

7. The physiological information processing method according to claim 5,
wherein the information on the first activity evaluation index includes a trend graph illustrating a temporal change in the first activity evaluation index, and
wherein the information on the second activity evaluation index includes a trend graph illustrating a temporal change in the second activity evaluation index.

8. The physiological information processing method according to claim 5,
wherein the first respiratory muscle is a diaphragm, and
wherein the second respiratory muscle is an intercostal muscle.

9. The physiological information processing method according to claim 8,
wherein the first electrode is attached in an intercostal space of the subject, and
wherein the second electrode is attached to limbs or a chest of the subject.

10. The physiological information processing method according to claim 1,
wherein the plurality of electrodes are used in a 12-lead electrocardiogram examination.

11. The physiological information processing method according to claim 5,
wherein the first electrode includes a lead electrode associated with a V4 lead and a lead electrode associated with a V5 lead, and
wherein the first activity evaluation index is acquired based on an electrocardiogram signal that is acquired from the lead electrode associated with the V4 lead and from which the one or more first waveform components have been removed and an electrocardiogram signal that is acquired from the lead electrode associated with the V5 lead and from which the one or more first waveform components have been removed.

12. The physiological information processing apparatus comprising:
one or more processors; and
one or more memories configured to store a computer readable command,
wherein, when the computer readable command is executed by the processor, the physiological information processing apparatus executes:
a step of acquiring, from a plurality of electrodes attached to a body surface of a subject, a plurality of electrocardiogram signals indicating an electrocardiogram waveform of the subject;
a step of separating, through independent component analysis, each of the plurality of electrocardiogram signals into one or more first waveform components indicating the electrocardiogram waveform and one or more second waveform components indicating a waveform other than the electrocardiogram waveform;
a step of acquiring an activity evaluation index indicating a respiratory muscle activity amount of the subject based on an electrocardiogram signal that is obtained from a predetermined electrode among the plurality of electrodes attached to the body surface of the subject and from which the one or more first waveform components have been removed; and
a step of outputting information on the activity evaluation index.

13. A non-transitory computer-readable storage medium that stores a program,
wherein the program causes a computer to execute:
a step of acquiring, from a plurality of electrodes attached to a body surface of a subject, a plurality of electrocardiogram signals indicating an electrocardiogram waveform of the subject;
a step of separating, through independent component analysis, each of the plurality of electrocardiogram signals into one or more first waveform components indicating the electrocardiogram waveform and one or more second waveform components indicating a waveform other than the electrocardiogram waveform;
a step of acquiring an activity evaluation index indicating a respiratory muscle activity amount of the subject based on an electrocardiogram signal that is obtained from a predetermined electrode among the plurality of electrodes attached to the body surface of the subject and from which the one or more first waveform components have been removed; and
a step of outputting information on the activity evaluation index.
